(19) United States
(12) Patent Application Publication
(10) Pub. No.: US 2021/0361234 A1
(43) Pub. Date: Nov. 25, 2021

(54) ORAL APPLIANCE FOR MEASUREMENT OF A TONGUE MOTION WITHIN A PATIENT'S ORAL CAVITY

(71) Applicant: Bam Medical, LTD., Tel Aviv (IL)

(72) Inventors: Eli DRAPISZ, Tel Aviv (IL); Eyal BOTZER, Ramat Hasharon (IL); Reuven MARKO, Netanya (IL)

(73) Assignee: Bam Medical, LTD, Tel Aviv (IL)

(21) Appl. No.: 17/324,468

(22) Filed: May 19, 2021

Related U.S. Application Data
(60) Provisional application No. 63/028,325, filed on May 21, 2020.

(51) Int. Cl. A61B 5/00; A61B 5/11

(52) U.S. Cl. CPC A61B 5/682; A61B 5/1126; A61B 5/4552

(57) ABSTRACT

Oral appliances and techniques for treating patients having abnormal tongue development using such oral appliances. An oral appliance includes a sheath adapted for placement in proximal to a tongue within a mouth of a patient, wherein a side of the sheath which comes into contact with the tongue when placed in the mouth substantially conforms at least in shape to features of the tongue; and at least one sensor embedded within the sheath, wherein each of the at least one sensor is configured to capture signals indicative of motion of the tongue.

### CROSS-REFERENCE TO RELATED APPLICATIONS

This application claims the benefit of U.S. Provisional Application No. 63/028,325 filed on May 21, 2020, the contents of which are hereby incorporated by reference.

### TECHNICAL FIELD

The disclosure generally relates to an oral appliance, and more particularly to an oral appliance for measuring motion parameters of a patient's tongue within the patient's oral cavity.

### BACKGROUND

Improper functioning of a patient's tongue may impact the patient's overall health in multiple ways. Such improper functioning may begin at infancy, causing issues with the patent's breast-feeding abilities, and can continue with conditions and ailments such as, but not limited to, craniofacial growth and development, improper breathing, insomnia, speech defects, and more. Some of the causes of less than optimal motion of the tongue within the oral cavity result from an excessively strict lingual frenulum or neurological or muscular tongue malfunction. The lingual frenulum, also referred to as the frenulum, is a mucous membrane that extends from the floor of the oral cavity to the underside of the tongue. In some cases, it extends so far out that it must be surgically detached, a condition known as ankyloglossia or tongue-tie. In other cases, such a disorder is not severe enough for surgery or is not timely diagnosed and therefore various conditions may develop that require diagnosis as well as therapy.

Various studies and solution have been proposed for the purpose of diagnosing proper motion of the tongue within the oral cavity. A paper by Elad, D. et al, titled “Biomechanics of Milk Extraction During Breast-Feeding” (hereinafter the “Paper”) checked the biomechanics of milk extraction to understand the motion of the infant's tongue during this process. The researchers used ultrasound to view the motion of the tongue versus the nipple as well as the floor and roof of the oral cavity which allowed the extraction of the tongue motion and was found to be a sinusoidal wave having a dominant frequency of approximately 1.56 Hz. See FIGS. 1 and 2 of the Paper. Based on this research, it is fair to assume that abnormalities in the sinus wave would be observed in the case where the tongue has substandard functionality. It should be realized, however, that the dominant frequency may have a range which is within an acceptable range of frequencies. Identifying such abnormalities using ultrasound for diagnostics, either on a breast-feeding mother or on other patients post lactation, is expensive and cumbersome.

Other solutions have been proposed. Some existing solutions involve the use of sensors that are attached to an oral appliance placed in the patient's mouth. Some of these solutions include a body that resembles an orthodontic retainer, and may further contain sensors that measure the motion of the tongue therein. The body at least partially lies against the upper palate of the oral cavity. However, such solutions face challenges at least because these solutions require a mouth that has teeth and because the size and shape of the body must be modified to fit each patient individually. Thus, these solutions cannot be applied to infants and may be inconvenient and costly to prepare for other patients. Further, the distance of the body that lies against the upper palate and the tongue of the patient may lead to improper measurements as the tongue never reaches close enough to the body and its sensors.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include an oral appliance. The oral appliance comprises: a sheath adapted for placement in proximal to a tongue within a mouth of a patient, wherein a side of the sheath which comes into contact with the tongue when placed in the mouth substantially conforms at least in shape to features of the tongue; and at least one sensor embedded within the sheath, wherein each of the at least one sensor is configured to capture signals indicative of motion of the tongue.

Certain embodiments disclosed herein also include a method for treating a patient having abnormal tongue development. The method comprises: determining, based on a plurality of electronic signals received from at least one sensor installed on an oral appliance inserted proximal to a tongue inside a mouth of the patient, at least one parameter of a dominant frequency of a motion of the tongue of the patient; determining, based on the determined at least one parameter, at least one status of the motion of the tongue of the patient; and providing a signal based on each of the at least one status.

Certain embodiments disclosed herein also include a non-transitory computer readable medium having stored thereon causing a processing circuitry to execute a process, the process comprising: determining, based on a plurality of electronic signals received from at least one sensor installed on an oral appliance inserted proximal to a tongue inside a mouth of the patient, at least one parameter of a dominant frequency of a motion of the tongue of the patient; determining, based on the determined at least one parameter, at least one status of the motion of the tongue of the patient; and providing a signal based on each of the at least one status.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is an illustration of an oral appliance for measurement of a tongue motion parameters within a patient's oral cavity according to an embodiment.

FIG. 2 is a schematic diagram of a controller of an oral appliance for measurement of a tongue motion parameters within a patient's oral cavity according to an embodiment.

FIG. 3A is a graph of a dominant frequency of a normally functioning tongue of an infant while breast-feeding generated according to an embodiment.

FIG. 3B is a graph of a dominant frequency of an abnormally functioning tongue of an infant while breast-feeding generated according to an embodiment.

FIG. 4 is a flowchart of a method for determining whether the tongue motion within the oral cavity is inside or outside of an expected sinus wave motion according to an embodiment.

FIG. 5 is an illustration of a cover to be fitted over the sheath portion of an oral appliance for measurement of a tongue motion parameters within a patient's oral cavity according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views. Descriptions of well-known parts are omitted for clarity.

The disclosed embodiments include oral appliances and methods for using oral appliances to measure tongue motion within a patient's oral cavity. A sheath is formed generally to conform to the tongue and is adapted to be inserted within the oral cavity above a patient's tongue. The bottom part of the sheath may substantially conform to the upper side of the Anterior and middle thirds of tongue. One or more sensors are embedded within the sheath. Such sensors may include, for example, sensors that measure pressure, acceleration, proximity, current, voltage, resistance, combinations thereof, and the like. Signals from the sensors may be used to determine motion parameters of the tongue within the oral cavity. In an embodiment, the sensors are communicatively connected to a controller that may provide at least partial processing of the signals received from the sensors. A wired or wireless interface may be provided for connecting the oral appliance and another user device such as a smart phone, a personal computer, a server, and the like.

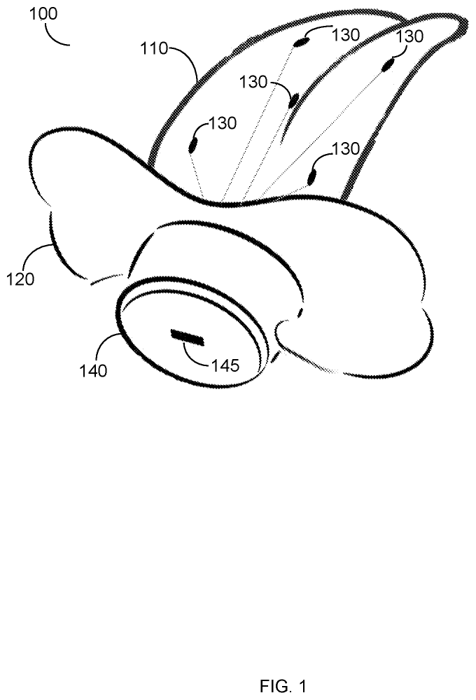

FIG. 1 is an illustration of an oral appliance 100 for measuring tongue motion parameters within a patient's oral cavity according to an embodiment. In an embodiment, the oral appliance 100 includes a sheath 110, a shield piece 120, one or more sensors 130, an enclosure 140, and a connector 145.

The sheath 110 is adapted for placement in a patient's mouth and deployment proximal to the patient's tongue. In an embodiment. the bottom side of the sheath, the one that comes into contact with the patient's tongue, substantially conforms in shape to the features of the tongue. In an embodiment, the sheath 110 substantially conforms in shape to the features of the tongue such that each of the sensors 130 comes into contact with a respective portion of the tongue, i.e., such that the sensors are deployed in a manner that allows for effective collection of data.

One of ordinary skill in the art would readily appreciate that while the sheath 110 is shown to have a particular shape, other shapes are possible for as long as they essentially conform to the upper side of the Anterior tongue. The shape and size of the sheath 110 may be based on a shape and size among common shapes and sizes of patients of different ages and/or of different sizes.

The sheath 110 is connected to the shield piece 120. The shield piece 120 adapted to prevent the patient using the oral device 100 from accidently swallowing the sheath 110. To this end, the shield piece 120 may be sized based on, for example, common mouth sizes of patients in different age ranges.

Into the sheath 110 there may be embedded one or more sensors 130. The sensors 130 may be sensors capable of measuring a variety of motion related parameters such as, but not limited to, pressure, acceleration, proximity, current, voltage, resistance sensors that provide one or more images, combinations thereof, and the like.

The sensors 130 are adapted to sense, measure or otherwise capture signals indicative of the motion parameters of the patient's tongue within the oral cavity. These signals may be processed so as to generate information regarding the motion parameters of the tongue within the oral cavity. The motion parameters provide indications that can be utilized to determine whether the tongue is functioning within an expected range. Performance within an expected range may require, as non-limiting examples, performing a tongue motion which is within a normal movement pattern, applying an expected pressure to the roof of the oral cavity, both, and the like. Normal movement patterns, expected pressures, and other measures of normal activity with respect to the parameters represented by the sensor signals may be predetermined (e.g., based on known normal parameters), may be learned via machine learning, and the like.

One of ordinary skill in the art would readily appreciate that, in order to measure certain of the parameters described above, sensors such as a gyro sensor, strain gage, accelerometer, and the like, may be used. In an embodiment, the shield piece 120 and the sheath 110 are detachable from each other, using a connecting member (not shown) that also provides therethrough connectivity to electronic circuitry (not shown in FIG. 1) embedded within the shield piece 120. This allows for disposing of the sheath 110 (e.g., after use in order to allow for replacing the sheath 110 before the next use) while reusing the shield piece 120.

In an embodiment, a connector 145 is communicatively connected to the sensors 130. The connector 145 may transfer the signals captured by the sensors 130 to a processing device (not shown) such as, but not limited to, a smart phone, a tablet, a personal computer, and the like. The signals may be transferred to such a device for processing, for example, as described herein.

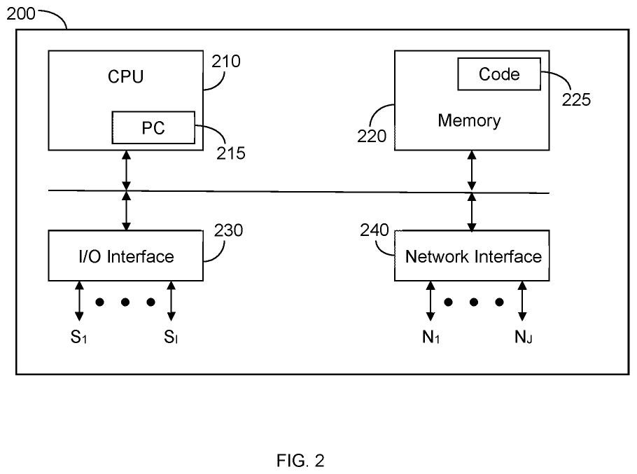

An enclosure 140 may be further used that contains therein circuitry connected to the connector 145 and the one or more sensors 130, the circuitry being used for at least partial processing of the signals received from the one or more sensors 130. In an embodiment, a display (not shown) may be mounted to the oral appliance 100, for example on the enclosure 140. The display may be communicatively connected to a controller (e.g., the controller 200, FIG. 2, not) for the purpose of displaying messages based on the parameters indicated by the signals collected by the sensors 130. The display may further comprise one or more light emitting diodes (LEDs) such as a red LED that represents a problem when lit, a green LED that represents that the tongue motion is within normal range when lit, a yellow LED that represents a less severe abnormality of the tongue motion within the oral cavity when lit, a combination thereof, and the like.

FIG. 2 is a schematic diagram of a controller 200 of an oral appliance for measurement of tongue motion parameters within a patient's oral cavity according to an embodiment. The controller 200 may provide for the circuitry described above with respect to FIG. 1.

The controller 210 includes a central processing unit (CPU) 210. The CPU 210 may be, but is not limited to, a microprocessor, a microcontroller, or other processing circuitry (PC) 215. The processing circuitry 215 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information

The CPU 210 is communicatively connected to a memory 220. The memory 220 may be volatile (e.g., random access memory, etc.), non-volatile (e.g., read only memory, flash memory, etc.), or a combination thereof. The memory 220 may store software to be executed by the processing circuitry of the CPU 210. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 215, configure the controller 200 to perform at least a portion of the various processes described herein. To this end, the memory 220 may further include a memory portion 225 dedicated for storing such instructions.

An input/output (I/O) interface 230 is also communicatively connected to the CPU 210 and provides connectivity to one or more sensors (e.g., the sensors 130, FIG. 1) through interfaces Si through Si, where 1′ is an integer equal to or greater than ‘1’. The I/O interface 230 may provide the connectivity between the one or more sensors 130 and the controller 200. The controller 200 may further comprise a network interface 240 that is communicatively connected to the CPU 210, and further provides interfaces to one or more networks using network interfaces N₁ through Nⱼ, where ‘j’ is an integer equal to or greater than ‘1’. Network interfaces may include, but are not limited to, wired or wireless connections, serial or parallel in nature, and the like. These may include, without limitations, a universal serial bus (USB), WiFi, near-field communication (NFC), and the like. In an embodiment, the code, when executed, causes the controller 200 to process the signals captured by the sensors 130 so as to establish the motion of the patient's tongue within the oral cavity.

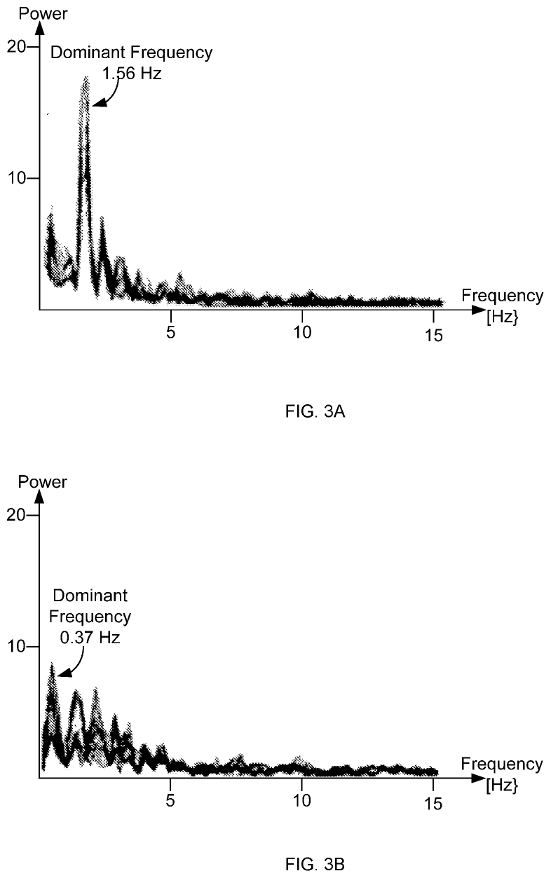

FIG. 3A and FIG. 3B provide example graphs of dominant frequencies in the case of normal tongue motion (e.g., the normal tongue motion discussed in the Paper) and abnormal tongue motion, respectively.

As shown in FIG. 3A, in a normally functioning tongue, the dominant frequency is expected to be approximately 1.56 Hz (which may differ from case to case within an acceptable range) and at a power having a value of roughly 18 (the power having a value corresponding to a pressure applied by the tongue). The secondary frequency, i.e., the next most dominant frequency, is a lower frequency at about 0.4 Hz and at a power of roughly 7, i.e., approximately two and a half times smaller. Each of the dominant frequency and the secondary frequency is a local peak, i.e., a point with a value that is greater than or equal to the value of each of its two neighboring points.

As shown in FIG. 3B, an abnormally functioning tongue will often present a dominant frequency that has a power level that is similar to that found for the secondary and tertiary frequencies. Therefore, it has been identified that it is possible to differentiate between a properly functioning tongue and an abnormally functioning tongue by measuring the dominant frequency or both the dominant frequency and the secondary frequency. Consequently, the disclosed embodiments provide an oral appliance which allows for such measurements as well as techniques for using such measurements to detect abnormal tongue function.

More specifically, detection of abnormal tongue function can be achieved by determining that the measured dominant frequency departs from the expected dominant frequency of a normally functioning tongue (e.g., by determining a difference between the measured dominant frequency and the expected or normal dominant frequency above a threshold). Alternatively or collectively, abnormal tongue function can be detected by comparing the dominant frequency with the secondary frequency, where it is expected that a normally functioning tongue will present a power ratio between the dominant frequency and the secondary frequency of the tongue's motion to be above a predetermined value, for example a ratio of 2:1 of dominant:secondary frequency. Any time this ratio is above the predetermined ratio then the tongue is functioning properly and any time it is below there is an abnormality. Furthermore, a second ratio threshold maybe determined, for example 1.5 that would determine a less severe condition then when the ratio is below that second ratio,

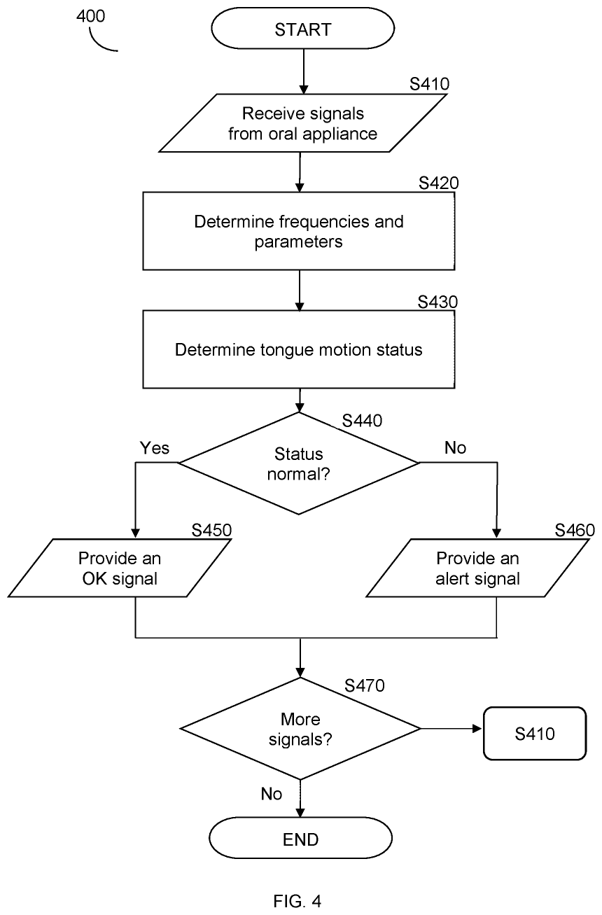

FIG. 4 is a flowchart 400 illustrating a method for determining whether the tongue motion within the oral cavity is inside or outside of an expected sinus wave motion according to an embodiment. The method of FIG. 4 may be utilized in order to treat a patient having an underdeveloped or otherwise abnormal tongue. In an embodiment, the method is used in conjunction with the oral appliance 100, FIG. 1, and may be used as a series of instructions performed by either a controller 200, FIG. 2, or a device communicatively connected to the oral apparatus 100. Such a device may be, for example, a smart phone.

At S410, signals are received from one or more sensors (e.g., the sensors 130 of the oral appliance 100).

At S420, frequencies, parameters, or both, of the tongue motion within the oral cavity are determined. In an embodiment, S420 includes processing the signals, using for example one or more digital signal processing (DSP) techniques and/or algorithms. The processing at least results in a dominant frequency, and may result in other frequencies and/or parameters.

In an embodiment, only the dominant frequency of the tongue motion parameter is determined. Alternatively, both the dominant frequency of the tongue motion parameter and at least a secondary frequency of the tongue motion parameter are determined.

In an embodiment, in addition to any determined frequencies, parameters that relate to the pressure applied by the tongue against the bottom surface of the sheath 110 and against the roof of the oral cavity may also be determined. Further, such determination may be made in whole or in part by dynamically analyzing images taken of the tongue in connection with other motion parameters gathered as described herein.

At S430, the parameters determined for the tongue motion are used to determine a tongue motion status of the patient's tongue relative to predetermined standards. The tongue motion status may include, but is not limited to, a value representing a difference between frequencies or power values, a binary value representing a status of either normal or abnormal, a ratio, a combination thereof, and the like.

In an embodiment, S430 includes comparing a frequency value representing the dominant frequency to a dominant frequency range (including a predetermined range of frequency values) expected for a normally functioning tongue. The range may be centered around a predetermined frequency (as a non-limiting example, 1.56 Hz) determined by research to be a dominant frequency for a properly functioning tongue. In such an embodiment, a status is normal when the dominant frequency is within the dominant frequency range and abnormal otherwise.

In another embodiment, the power corresponding to the dominant frequency (i.e., the power indicated by signals captured at the same time as the signals based on which the dominant frequency was determined) is also checked to determine whether the value of the power is above a predetermined power value. As noted above, such power may have a value determined based on and corresponding to, for example but not limited to, a pressure applied by the tongue (e.g., a pressure based on a signal captured by a pressure sensor among the sensors 130, FIG. 1). The motion of the patient's tongue may be determined to be normal when the power value is greater than or equal to the predetermined power value, and abnormal otherwise.

In yet another embodiment, the power ratio between the dominant frequency and at least a secondary frequency may be determined. As noted above, there should be a significant difference (e.g., above a predetermined threshold) between these two powers for a normally functioning tongue. If the difference is not above a threshold, the tongue motion status is determined as abnormal.

For any of the thresholds utilized for detecting abnormal tongue motion statuses, different thresholds may be utilized to define different severities of abnormality. In this regard, it is noted that abnormalities in tongue development do not all have the same severity, and that different severities of abnormal tongue development may warrant different treatments or other interventions. As a non-limiting example, more severe abnormalities may require corrective surgery, while less severe abnormalities may only require exercises or other minor corrections which are less risky, invasive, and/or expensive.

In other embodiments, the tongue motion status may be determined using a machine learning model trained based on training frequencies, training power values, or both. Such a machine learning model may be configured to, for example, classify values as normal or abnormal, detect abnormalities in frequency differences, and the like. The training data may be frequencies and/or power values of prior patients, and different models may be trained based on patients in different categories (e.g., categories defined with respect to age) that may be associated with different frequency and/or power relationships.

At S440, based on the tongue motion status, it is determined whether the status of the tongue is normal. If so, execution continues with S450; otherwise, execution continues with S460. As noted above, such a status may be normal if a particular value is within a threshold difference from another value, when a ratio between values is above a threshold, both, and the like.

At S450, when the status is determined as normal, an “OK” signal may be provided. Such a signal may be, but is not limited to, an electronic message, an LED lit in green, a display showing the term “OK,” turning a light source to an “ON” position (i.e., such that the light source emits light), combinations thereof and the like.

At S460, an alert signal indicating that there is a problem is provided. Such an alert signal may be, but is not limited to, an electronic message, an LED lit in red, a display providing an indication of a problem, turning a light source to an “ON” position (i.e., such that the light source does not emit light), and the like. In some embodiments, different indicators may be used based on the severity of the abnormality. As a non-limiting example, a yellow LED may be used to indicate a less severe abnormality while a red LED may be used to indicate a more severe abnormality.

At S470, it is determined whether additional signals have been received and, if so, execution continues with S410; otherwise, execution terminates.

In this regard, it is noted that repeatedly checking tongue motion status for abnormalities and alerting the patient or a medical professional of such abnormalities may be utilized to train the patient as part of a treatment process. More specifically, such training may involve having the patient perform exercises designed to demonstrate proper or improper tongue motion, and the patient can be instructed on how to improve their tongue's motions based on the signals provided during the exercises. In other words, the signals provided as described herein may be utilized as feedback so that the patient may understand or otherwise comprehend when the tongue is used within normal or otherwise expected ranges and when it is not. The training may be provided for the use of the tongue in its entirety or any portion thereof. Accordingly, such training allows for treating a patient in order to improve their tongue functions.

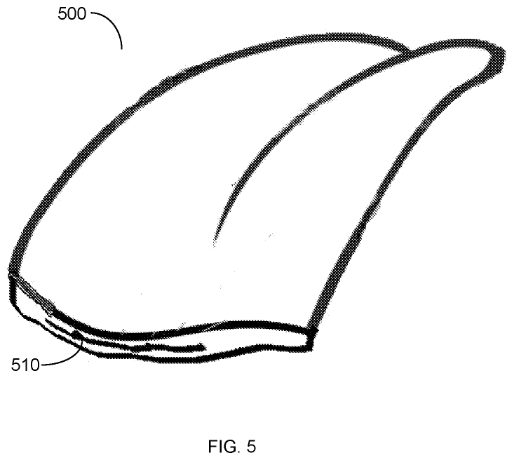

FIG. 5 is an example illustration of a cover 500 to be fitted over the sheath portion of an oral appliance for measurement of a tongue motion parameters within a patient's oral cavity according to an embodiment. The cover 500 defines a cavity (not shown) and an aperture such as a slit 510 through which a sheath (e.g., the sheath 110 of the oral appliance 100) is inserted therethrough into the hollow of the cover 500. The cover 500 is adapted to tightly correspond to the sheath 110 when applied thereto such that, when the cover 500 is inserted into a patient's oral cavity, the cover 500 cannot be easily detached from the sheath and accidently be swallowed by a patient.

The cover 500 may be made of, for example but not limited to, thin silicon. The cover 500 may be adapted to expand around the sheath 110 to closely engage therewith. In an embodiment, the cover 500 is sufficiently thin so as to allow the various sensors 130 to properly operate without tearing due to normal stresses applied by the tongue when within the patient's oral cavity. Upon completion of the measurements, the cover 500 may be removed from the sheath 110 and disposed thereof, allowing for practical reuse of the oral device 100.

The various processes disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. An oral appliance, comprising:
a sheath adapted for placement in proximal to a tongue within a mouth of a patient, wherein a side of the sheath which comes into contact with the tongue when placed in the mouth substantially conforms at least in shape to features of the tongue; and
at least one sensor embedded within the sheath, wherein each of the at least one sensor is configured to capture signals indicative of motion of the tongue.

2. The oral appliance of claim 1, further comprising:
a shield connected to the sheath, wherein the shield is adapted to prevent swallowing of the oral appliance.

3. The oral appliance of claim 2, wherein the shield is detachable from the sheath.

4. The oral appliance of claim 1, further comprising:
a connector communicatively connected to each of the at least one sensor, wherein the connector is configured to provide electrical connectivity to each of the at least one sensor.

5. The oral appliance of claim 1, further comprising:
an input/output interface communicatively connected to the at least one sensor;
a processing circuitry communicatively connected to the input/output interface; and
a memory communicatively connected to the processing circuitry, wherein the memory contains instructions that, when executed by the processing circuitry, configures the processing circuitry to process the signals captured by the at least one sensor.

6. The oral appliance of claim 5, wherein the memory further contains instructions that, when executed by the processing circuitry, configures the processing circuitry to:
determine at least one dominant frequency based on the signals captured by the at least one sensor, wherein each of the at least one dominant frequency is a first local peak among a respective plurality of local peaks represented by the signals captured by the sensor; and
determine, for each of the at least one dominant frequency, whether a respective portion of the motion of the tongue is normal based on the dominant frequency.

7. The oral appliance of claim 5, wherein the memory further contains instructions that, when executed by the processing circuitry, configures the processing circuitry to:
determine at least one value of power exerted by the tongue; and
determine, for each of the at least one value of power, whether a respective portion of the motion of the tongue is normal based on the value of power.

8. The oral appliance of claim 1, wherein the at least one sensor includes at least one of: a pressure sensor, an acceleration sensor, a proximity sensor, a current sensor, a voltage sensor, a resistance sensor, and an image sensor.

9. The oral appliance of claim 1, further comprising:
a cover defining a cavity and an aperture, wherein the cover is adapted to allow for insertion of the sheath through the aperture and into the cavity.

10. A method for treating a patient having abnormal tongue development, comprising:
determining, based on a plurality of electronic signals received from at least one sensor installed on an oral appliance inserted proximal to a tongue inside a mouth of the patient, at least one parameter of a dominant frequency of a motion of the tongue of the patient;
determining, based on the determined at least one parameter, at least one status of the motion of the tongue of the patient; and
providing a signal based on each of the at least one status.

11. The method of claim 10, wherein the at least one parameter of each dominant frequency includes a frequency value of the dominant frequency, wherein determining the status for a dominant frequency of the at least one dominant frequency further comprises:
comparing a predetermined range of frequency values to the frequency value of the dominant frequency, wherein the status is determined as normal when the frequency value is within the predetermined range of frequency values.

12. The method of claim 10, wherein the at least one parameter of each dominant frequency includes a power value corresponding to the dominant frequency, wherein determining the status for a dominant frequency of the at least one dominant frequency further comprises:
comparing a predetermined power value with the power value corresponding to the dominant frequency, wherein the status is determined as normal when the power value corresponding to the dominant frequency is greater than or equal to the predetermined power value.

13. The method of claim 10, wherein the at least one parameter of each dominant frequency includes a first frequency value of the dominant frequency and a second frequency value of a secondary frequency, wherein determining the status for a dominant frequency of the at least one dominant frequency further comprises:
determining a ratio between the dominant frequency and the secondary frequency based on the first frequency value and the second frequency value, wherein the status is determined as normal when the determined ratio is above a threshold.

14. The method of claim 10, wherein each provided signal indicates either normal motion or abnormal motion.

15. The method of claim 14, wherein the at least one signal is a plurality of signals, wherein the plurality of signals is provided as feedback in response to motion of the tongue of the patient during an exercise.

16. The method of claim 14, wherein the patient is instructed based on the feedback in order to treat at least one abnormality in tongue development of the patient.

17. The method of claim 10, wherein the oral appliance oral appliance includes a sheath and at least one sensor embedded within the sheath, wherein a side of the sheath which comes into contact with the tongue when placed in the mouth substantially conforms at least in shape to features of the tongue, wherein each of the at least one sensor is configured to capture signals indicative of motion of the tongue.

18. A non-transitory computer readable medium having stored thereon instructions for causing a processing circuitry to execute a process, the process comprising:
determining, based on a plurality of electronic signals received from at least one sensor installed on an oral appliance inserted proximal to a tongue inside a mouth of the patient, at least one parameter of a dominant frequency of a motion of the tongue of the patient;
determining, based on the determined at least one parameter, at least one status of the motion of the tongue of the patient; and
providing a signal based on each of the at least one status.
